(19) United States
(12) Patent Application Publication
(10) Pub. No.: US 2006/0073133 A1
(43) Pub. Date: Apr. 6, 2006

(54) Sialoadhesin factor-3 antibodies

(76) Inventors: Kristine Kay Kikly, Linfield, PA (US); Julie A. Abrahamson, Essex (GB)

Correspondence Address: GLAXOSMITHKLINE;Corporate Intellectual Property - UW2220, P.O. Box 1539, King of Prussia, PA, 19406-0939, US

(21) Appl. No.: 10/246,019

(22) Filed: Sep. 18, 2002

Related U.S. Application Data
(63) Continuation of application No. 09/577,930, filed on May 24, 2000, now abandoned.
Application No. 09/577,930 is a continuation-in-part of application No. 09/046,736, filed on Mar. 24, 1998, now Pat. No. 6,090,582.
(60) Provisional application No. 60/041,885, filed on Apr. 2, 1997.

(51) Int. Cl. A61K 39/395; C07H 21/04; C12P 21/06; C12N 5/06

(52) U.S. Cl. 424/130.1; 530/387.1; 435/69.1; 435/320.1; 435/326; 536/23.53

(57) ABSTRACT

Monoclonal antibodies have been generated that bind to human sialoadhesion factor-3. These antibodies are useful as diagnostic and therapeutic reagents.

This application is a continuation-in-part of application Ser. No. 09/046,736, filed Mar. 24, 1998, which claims the benefit of U.S. Provisional Application No. 60/041,885, filed Apr. 2, 1997.

### FIELD OF THE INVENTION

This invention relates to monoclonal antibodies (mAbs) that bind to sialoadhesin factor-3 (SAF-3), and to the use of such antibodies for diagnostic and therapeutic purposes.

### BACKGROUND OF THE INVENTION

The drug discovery process is currently undergoing a fundamental revolution as it embraces “functional genomics”, that is, high throughput genome- or gene-based biology. This approach is rapidly superceding earlier approaches based on “positional cloning”. A phenotype, that is a biological function or genetic disease, would be identified and this would then be tracked back to the responsible gene, based on its genetic map position.

Functional genomics relies heavily on the various tools of bioinformatics to identify gene sequences of potential interest from the many molecular biology databases now available. There is a continuing need to identify and characterise further genes and their related polypeptides/proteins, as targets for drug discovery.

### SUMMARY OF THE INVENTION

The present invention relates to SAF-3, in particular SAF-3 polypeptides and SAF-3 polynucleotides, recombinant materials and methods for their production. In another aspect, the invention relates to methods for using such polypeptides and polynucleotides, including the treatment of cancer, inflammation, autoimmunity, allergy, asthma, rheumatoid arthritis, CNS inflammation, multiple sclerosis, AIDS, and bacterial, fungal, protozoan and viral infections, hereinafter referred to as “the Diseases”, amongst others. In a further aspect, the invention relates to methods for identifying agonists and antagonists/inhibitors using the materials provided by the invention, and treating conditions associated with SAF-3 imbalance with the identified compounds. In a still further aspect, the invention relates to diagnostic assays for detecting diseases associated with inappropriate SAF-3 activity or levels.

Yet another aspect of the present invention includes a monoclonal antibody that binds to human SAF-3. More specifically, the present invention includes a monoclonal antibody having the identifying characteristics of monoclonal antibody that is a member selected from the group consisting of 12B1, 2H10, 2G4, 7D9, 13H5, 16F2, 13D5, 16D3 and 12E7. Preferred is an antibody comprising a heavy chain variable region polypeptide as set forth in SEQ ID NO:6 and a kappa light chain variable region polypeptide as set forth in SEQ ID NO:8.

The present invention also includes an immunoglobulin heavy chain complementarity determining region comprising any of the polypeptides set forth in SEQ ID NOs:9, 10 or 11 or any combination thereof, and an immunoglobulin kappa light chain complementarity determining region comprising any of the polypeptides set forth in SEQ ID NOs:12, 13 or 14 or any combination thereof. A preferred embodiment of the present invention is a polypeptide comprising an immunoglobulin complementarity determining region comprising the polypeptides set forth in SEQ ID NO:9, 10, 11, 12, 13 and 14. The present invention also includes an isolated polynucleotide encoding any of the forgoing polypeptides.

Another aspect to the present invention includes a method for treating or preventing various disease states in a mammal including cancer, inflammation, autoimmunity, allergy, asthma, rheumatoid arthritis, CNS inflammation, multiple sclerosis, AIDS, and bacterial, fungal, protozoan and viral infections comprising administering to a subject in need thereof an effective dose of a monoclonal antibody against human SAF-3.

Yet another aspect of the present invention includes a pharmaceutical composition comprising a monoclonal antibody against human SAF-3.

### BRIEF DESCRIPTION OF THE DRAWINGS

FIG. 1 shows the V_{H} cDNA sequence and the deduced amino acid sequence of a monoclonal antibody that binds to SAF-3, mAb 12B1 (SEQ ID NOs:5 and 6, respectively). The bolded residues indicate the three CDRs (SEQ ID NOs:9, 10, and 11).

FIG. 2 shows the V_{K} cDNA sequence and the deduced amino acid sequence of a monoclonal antibody that binds to SAF-3, 12B1 (SEQ ID NOs:7 and 8, respectively). The bolded residues indicate the three CDRs (SEQ ID NOs:12, 13, and 14).

### DESCRIPTION OF THE INVENTION

In a first aspect, the present invention relates to SAF-3 polypeptides. Such peptides include isolated polypetides comprising an amino acid sequence which has at least 70% identity, preferably at least 80% identity, more preferably at least 90% identity, yet more preferably at least 95% identity, most preferably at least 97-99% identity, to that of SEQ ID NO:2 over the entire length of SEQ ID NO:2. Such polypeptides include those comprising the amino acid of SEQ ID NO:2.

Further peptides of the present invention include isolated polypeptides in which the amino acid sequence has at least 70% identity, preferably at least 80% identity, more preferably at least 90% identity, yet more preferably at least 95% identity, most preferably at least 97-99% identity, to the amino acid sequence of SEQ ID NO:2 over the entire length of SEQ ID NO:2. Such polypeptides include the polypeptide of SEQ ID NO:2.

Further peptides of the present invention include isolated polypeptides encoded by a polynucleotide comprising the sequence contained in SEQ ID NO:1.

Polypeptides of the present invention are believed to be members of the sialoadhesin family of polypeptides. They are therefore of interest because the sialoadhesin family of proteins, sialoadhesin, CD33, CD22 and myelin-associated glycoprotein (MAG), are utilized as cellular interaction molecules. They bind specific carbohydrates in a sialic acid dependent manner on target cells. The extracellular domain is made up of various numbers of immunoglobulin-like domains of the V-like and C2-like subtypes and the intracellular portion has no known homology to any signalling motifs. Sialoadhesin expression is restricted to macrophages, it has 17 Ig-like domains and the specific recognition sequence on target cells is Neu5Acα2,3Galβ13GalNAc. Known target cells are developing myeloid cells in the bone marrow and lymphocytes in the spleen and lymph node (Crocker, P. R., et al. (1994) EMBO J. 13:4490-4503). CD22 is expressed only on B cells and has α and β isoforms with 5 and 7 Ig-like domains, respectively. CD22 is known to bind T cells, B cells, monocytes, granulocytes and erythrocytes by recognizing Neu5Acα2,6Galβ1,4Glc(NAc) in N-linked glycans (Crocker, P. R., et al. (1994) EMBO J. 13:4490-4503; Stamenkovic, I. and Seed, B. (1990) Nature 345:74-77; Wilson, G. L., et al. (1991) J Exp Med 173:137-146). Myelin-associated glycoprotein (MAG) is expressed by Schwann cells in the peripheral nervous system and oligodendrocytes in the central nervous system and is thought to participate in the cell adhesion to axons. MAG has two alternatively spliced variants, large MAG (L-MAG) and small MAG (S-MAG) which are expressed either during embryonic development or in the adult, respectively. The alternative splicing results in the expression of the same extracellular domains but distinct intracellular domains (Pedraza, L. et al. (1990) JCB 111:2651-2661).

CD33 is most relevant to SAF-3 because they are the most closely related of all the family members. CD33 is normally expressed on the developing myelomonocytic lineage. It is absent on early stem cells but is present on colony-forming units for granulocytes, erythrocytes, monocytes, and megakaryocytes (CFU-GEMM) and progenitors of granulocytes and mononuclear phagocytes (CFU-GM). It is downregulated by mature granulocytes but retained by mature monocytes and macrophages (Andrews, R. G., et al. (1983) Blood 62:124; Griffin, J. D., et al. (1984) Leuk Res 8:521). CD33 has two Ig-like domains and prefers to bind targets expressing NeuAcα2,3Gal in N- and O-linked glycans. It maps to chromosome 19q13.1-13.3, closely linking it in the genome with MAG and CD22 (Freeman, S. D., et al. (1995) Blood 85:2005-2012).

CD33 has also been found to be expressed on about 85% of leukemic myeloblasts in patients with acute myelogenous leukemia (AML) and is frequently used to differentiate AML from acute lymphoblastic leukemia (ALL). Monoclonal antibodies to CD33 have been used therapeutically to purge residual myeloblasts from autologous bone marrow grafts ex vivo for the treatment of AML (Robertson, M. J., et al. (1992) Blood 79:2229-2236). More recently, humanized monoclonal antibodies to CD33 have undergone evaluation in vivo for the treatment of AML (Caron, P. C., et al. (1994) Blood 83:1760-1768). These properties are hereinafter referred to as “SAF-3 activity” or “SAF-3 polypeptide activity” or “biological activity of SAF-3”. Also included amongst these activities are antigenic and immunogenic activities of said SAF-3 polypeptides, in particular the antigenic and immunogenic activities of the polypeptide of SEQ ID NO:2. Preferably, a polypeptide of the present invention exhibits at least one biological activity of SAF-3.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes include variants of the aforementioned polypetides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

In a further aspect, the present invention relates to SAF-3 polynucleotides. Such polynucleotides include isolated polynucleotides comprising a nucleotide sequence encoding a polypeptide which has at least 70% identity, preferably at least 80% identity, more preferably at least 90% identity, yet more preferably at least 95% identity, to the amino acid sequence of SEQ ID NO:2, over the entire length of SEQ ID NO:2. In this regard, polypeptides which have at least 97% identity are highly preferred, whilst those with at least 98-99% identity are more highly preferred, and those with at least 99% identity are most highly preferred. Such polynucleotides include a polynucleotide comprising the nucleotide sequence contained in SEQ ID NO:1 encoding the polypeptide of SEQ ID NO:2.

Further polynucleotides of the present invention include isolated polynucleotides comprising a nucleotide sequence that has at least 70% identity, preferably at least 80% identity, more preferably at least 90% identity, yet more preferably at least 95% identity, to a nucleotide sequence encoding a polypeptide of SEQ ID NO:2, over the entire coding region. In this regard, polynucleotides which have at least 97% identity are highly preferred, whilst those with at least 98-99% identity are more highly preferred, and those with at least 99% identity are most highly preferred.

Further polynucleotides of the present invention include isolated polynucleotides comprising a nucleotide sequence which has at least 70% identity, preferably at least 80% identity, more preferably at least 90% identity, yet more preferably at least 95% identity, to SEQ ID NO:1 over the entire length of SEQ ID NO:1. In this regard, polynucleotides which have at least 97% identity are highly preferred, whilst those with at least 98-99% identity are more highly preferred, and those with at least 99% identity are most highly preferred. Such polynucleotides include a polynucleotide comprising the polynucleotide of SEQ ID NO:1 as well as the polynucleotide of SEQ ID NO:1.

The invention also provides polynucleotides which are complementary to all the above described polynucleotides.

The nucleotide sequence of SEQ ID NO:1 shows homology with CD33 (Simmons, D., and Seed, B. (1988) J. Immunology 141:2797-2800). The nucleotide sequence of SEQ ID NO: 1 is a cDNA sequence and comprises a polypeptide encoding sequence (nucleotide 23 to 1426) encoding a polypeptide of 467 amino acids, the polypeptide of SEQ ID NO:2. The nucleotide sequence encoding the polypeptide of SEQ ID NO:2 may be identical to the polypeptide encoding sequence contained in SEQ ID NO:1 or it may be a sequence other than the one contained in SEQ ID NO:1, which, as a result of the redundancy (degeneracy) of the genetic code, also encodes the polypeptide of SEQ ID NO:2. The polypeptide of the SEQ ID NO:2 is structurally related to other proteins of the sialoadhesin family, having homology and/or structural similarity with CD33 (Simmons, D., and Seed, B. (1988) J. Immunology 141:2797-2800).

Preferred polypeptides and polynucleotides of the present invention are expected to have, inter alia, similar biological functions/properties to their homologous polypeptides and polynucleotides. Furthermore, preferred polypeptides and polynucleotides of the present invention have at least one SAF-3 activity.

The present invention also relates to partial or other polynucleotide and polypeptide sequences which were first identified prior to the determination of the corresponding full length sequences of SEQ ID NO:1 and SEQ ID NO:2.

Accordingly, in a further aspect, the present invention provides for an isolated polynucleotide comprising:

(a) a nucleotide sequence which has at least 70% identity, preferably at least 80% identity, more preferably at least 90% identity, yet more preferably at least 95% identity, even more preferably at least 97-99% identity to SEQ ID NO:3 over the entire length of SEQ ID NO:3;

(b) a nucleotide sequence which has at least 70% identity, preferably at least 80% identity, more preferably at least 90% identity, yet more preferably at least 95% identity, even more preferably at least 97-99% identity, to SEQ ID NO:3 over the entire length of SEQ ID NO:3;

(c) the polynucleotide of SEQ ID NO:3; or

(d) a nucleotide sequence encoding a polypeptide which has at least 70% identity, preferably at least 80% identity, more preferably at least 90% identity, yet more preferably at least 95% identity, even more preferably at least 97-99% identity, to the amino acid sequence of SEQ ID NO:4, over the entire length of SEQ ID NO:4; as well as the polynucleotide of SEQ ID NO:3.

The present invention further provides for a polypeptide which:

(a) comprises an amino acid sequence which has at least 70% identity, preferably at least 80% identity, more preferably at least 90% identity, yet more preferably at least 95% identity, most preferably at least 97-99% identity, to that of SEQ ID NO:4 over the entire length of SEQ ID NO:4;

(b) has an amino acid sequence which is at least 70% identity, preferably at least 80% identity, more preferably at least 90% identity, yet more preferably at least 95% identity, most preferably at least 97-99% identity, to the amino acid sequence of SEQ ID NO:4 over the entire length of SEQ ID NO:4;

(c) comprises the amino acid of SEQ ID NO:4; and

(d) is the polypeptide of SEQ ID NO:4; as well as polypeptides encoded by a polynucleotide comprising the sequence contained in SEQ ID NO:3.

The nucleotide sequence of SEQ ID NO:3 and the peptide sequence encoded thereby are derived from EST (Expressed Sequence Tag) sequences. It is recognized by those skilled in the art that there will inevitably be some nucleotide sequence reading errors in EST sequences (see Adams, M. D. et al. (1995) Nature 377 (supp): 3). Accordingly, the nucleotide sequence of SEQ ID NO:3 and the peptide sequence encoded therefrom are therefore subject to the same inherent limitations in sequence accuracy. Furthermore, the peptide sequence encoded by SEQ ID NO:3 comprises a region of identity or close homology and/or close structural similarity (for example a conservative amino acid difference) with the closest homologous or structurally similar protein.

Polynucleotides of the present invention may be obtained, using standard cloning and screening techniques, from a cDNA library derived from mRNA in cells of human myleoid lineage, using the expressed sequence tag (EST) analysis (Adams, M. D. et al. (1991) Science 252:1651-1656; Adams, M. D. et al. (1992) Nature, 355:632-634; Adams, M. D., et al. (1995) Nature 377 Supp:3-174). Polynucleotides of the invention can also be obtained from natural sources such as genomic DNA libraries or can be synthesized using well known and commercially available techniques.

When polynucleotides of the present invention are used for the recombinant production of polypeptides of the present invention, the polynucleotide may include the coding sequence for the mature polypeptide, by itself; or the coding sequence for the mature polypeptide in reading frame with other coding sequences, such as those encoding a leader or secretory sequence, a pre-, or pro- or prepro-protein sequence, or other fusion peptide portions. For example, a marker sequence which facilitates purification of the fused polypeptide can be encoded. In certain preferred embodiments of this aspect of the invention, the marker sequence is a hexa-histidine peptide, as provided in the pQE vector (Qiagen, Inc.) and described in Gentz et al. (1989) Proc Natl Acad Sci USA 86:821-824, or is an HA tag. The polynucleotide may also contain non-coding 5′ and 3′ sequences, such as transcribed, non-translated sequences, splicing and polyadenylation signals, ribosome binding sites and sequences that stabilize mRNA.

Further embodiments of the present invention include polynucleotides encoding polypeptide variants which comprise the amino acid sequence of SEQ ID NO:2 and in which several, for instance from 5 to 10, 1 to 5, 1 to 3, 1 to 2 or 1, amino acid residues are substituted, deleted or added, in any combination.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1. Typically these nucleotide sequences are 70% identical, preferably 80% identical, more preferably 90% identical, most preferably 95% identical to that of the referent. The probes or primers will generally comprise at least 15 nucleotides, preferably, at least 30 nucleotides and may have at least 50 nucleotides. Particularly preferred probes will have between 30 and 50 nucleotides.

A polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO:1 or a fragment thereof; and isolating full-length cDNA and genomic clones containing said polynucleotide sequence. Such hybridization techniques are well known to the skilled artisan. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Thus the present invention also includes polynucleotides obtainable by screening an appropriate library under stingent hybridization conditions with a labeled probe having the sequence of SEQ ID NO:1 or a fragment thereof.

The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low ‘processivity’ (a measure of the ability of the enzyme to remain attached to the template during the polymerisation reaction), failing to complete a DNA copy of the mRNA template during 1st strand cDNA synthesis.

There are several methods available and well known to those skilled in the art to obtain full-length cDNAs, or extend short cDNAs, for example those based on the method of Rapid Amplification of cDNA ends (RACE) (see, for example, Frohman et al. (1988) PNAS USA 85: 8998-9002). Recent modifications of the technique, exemplified by the Marathon™′ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the ‘missing’ 5′ end of the cDNA using a combination of gene specific and adaptor specific oligonucleotide primers. The PCR reaction is then repeated using ‘nested’ primers, that is, primers designed to anneal within the amplified product (typically an adaptor specific primer that anneals further 3′ in the adaptor sequence and a gene specific primer that anneals further 5′ in the known gene sequence). The products of this reaction can then be analysed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems which comprise a polynucleotide or polynucleotides of the present invention, to host cells which are genetically engineered with such expression sytems and to the production of polypeptides of the invention by recombinant techniques. Cell-free translation systems can also be employed to produce such proteins using RNAs derived from the DNA constructs of the present invention.

For recombinant production, host cells can be genetically engineered to incorporate expression systems or portions thereof for polynucleotides of the present invention. Introduction of polynucleotides into host cells can be effected by methods described in many standard laboratory manuals, such as Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). Preferred such methods include, for instance, calcium phosphate transfection, DEAE-dextran mediated transfection, transvection, microinjection, cationic lipid-mediated transfection, electroporation, transduction, scrape loading, ballistic introduction or infection.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al. (supra). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

If a polypeptide of the present invention is to be expressed for use in screening assays, it is generally preferred that the polypeptide be produced at the surface of the cell. In this event, the cells may be harvested prior to use in the screening assay. If the polypeptide is secreted into the medium, the medium can be recovered in order to recover and purify the polypeptide. If produced intracellularly, the cells must first be lysed before the polypeptide is recovered.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and or purification.

This invention also relates to the use of polynucleotides of the present invention as diagnostic reagents. Detection of a mutated form of the gene characterized by the polynucleotide of SEQ ID NO:1 which is associated with a dysfunction will provide a diagnostic tool that can add to, or define, a diagnosis of a disease, or susceptibility to a disease, which results from under-expression, over-expression or altered expression of the gene. Individuals carrying mutations in the gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled SAF-3 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing (ee, e.g., Myers et al. (1985) Science 230:1242). Sequence changes at specific locations may also be revealed by nuclease protection assays, such as RNase and S1 protection or the chemical cleavage method (see Cotton et al. (1985) Proc Natl Acad Sci USA 85: 4397-4401). In another embodiment, an array of oligonucleotides probes comprising SAF-3 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al. (1996) Science 274:610-613).

The diagnostic assays offer a process for diagnosing or determining a susceptibility to the Diseases through detection of mutation in the SAF-3 gene by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays and flow cytometric analysis.

Thus in another aspect, the present invention relates to a diagonostic kit which comprises:

(a) a polynucleotide of the present invention, preferably the nucleotide sequence of SEQ ID NO: 1, or a fragment thereof;

(b) a nucleotide sequence complementary to that of (a);

(c) a polypeptide of the present invention, preferably the polypeptide of SEQ ID NO:2 or a fragment thereof; or

(d) an antibody to a polypeptide of the present invention, preferably to the polypeptide of SEQ ID NO:2.

It will be appreciated that in any such kit, (a), (b), (c) or (d) may comprise a substantial component. Such a kit will be of use in diagnosing a disease or suspectability to a disease, particularly cancer, inflammation, autoimmunity, allergy, asthma, rheumatoid arthritis, CNS inflammation, multiple sclerosis, AIDS, and bacterial, fungal, protozoan and viral infections, amongst others.

The nucleotide sequences of the present invention are also valuable for chromosome identification. The sequence is specifically targeted to, and can hybridize with, a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationship between genes and diseases that have been mapped to the same chromosomal region are then identified through linkage analysis (coinheritance of physically adjacent genes).

The differences in the cDNA or genomic sequence between affected and unaffected individuals can also be determined. If a mutation is observed in some or all of the affected individuals but not in any normal individuals, then the mutation is likely to be the causative agent of the disease.

In a further aspect, the present invention relates to genetically engineered soluble fusion proteins comprising a polypeptide of the present invention, or a fragment thereof, and various portions of the constant regions of heavy or light chains of immunoglobulins of various subclasses (IgG, IgM, IgA, IgE). Preferred as an immunoglobulin is the constant part of the heavy chain of human IgG, particularly IgG1, where fusion takes place at the hinge region. In a particular embodiment, the Fe part can be removed simply by incorporation of a cleavage sequence which can be cleaved with blood clotting factor Xa. Furthermore, this invention relates to processes for the preparation of these fusion proteins by genetic engineering, and to the use thereof for drug screening, diagnosis and therapy. In another approach, soluble forms of SAF-3 polypeptides still capable of binding the ligand in competition with endogenous SAF-3 may be administered. Typical embodiments of such competitors comprise fragments of the SAF-3 polypeptide. One example is using the extracellular domain of SAF-3 fused to a human immunoglobulin Fe region which could then be employed to treat cancer, inflammation, autoimmunity and allergy, among others. SAF-3/Fc polypeptides may also be employed to purge bone marrow ex vivo of cancer cells expressing SAF-3 ligands, as a tool to aid in the ex vivo expansion (proliferation and/or differentiation) of hematopoietic progenitor cells expressing SAF-3 ligands, as a stimulus in vivo for stem cell mobilization into the periphery, and as an in vivo chemoprotective agent. A further aspect of the invention also relates to polynucleotides encoding such fusion proteins. Examples of fusion protein technology can be found in International Patent Application Nos. WO94/29458 and WO94/22914.

Another aspect of the invention relates to a method for inducing an immunological response in a mammal which comprises inoculating the mammal with a polypeptide of the present invention, adequate to produce antibody and/or T cell immune response to protect said animal from the Diseases hereinbefore mentioned, amongst others. Yet another aspect of the invention relates to a method of inducing immunological response in a mammal which comprises, delivering a polypeptide of the present invention via a vector directing expression of the polynucleotide and coding for the polypeptide in vivo in order to induce such an immunological response to produce antibody to protect said animal from diseases.

A further aspect of the invention relates to an immunological/vaccine formulation (composition) which, when introduced into a mammalian host, induces an immunological response in that mammal to a polypeptide of the present invention wherein the composition comprises a polypeptide or polynucleotide of the present invention. The vaccine formulation may further comprise a suitable carrier. Since a polypeptide may be broken down in the stomach, it is preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation instonic with the blood of the recipient; and aqueous and non-aqueous sterile suspensions which may include suspending agents or thickening agents. The formulations may be presented in unit-dose or multi-dose containers, for example, sealed ampoules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Polypeptides of the present invention are responsible for many biological functions, including many disease states, in particular the Diseases hereinbefore mentioned. It is therefore desirous to devise screening methods to identify compounds which stimulate or which inhibit the function of the polypeptide. Accordingly, in a further aspect, the present invention provides for a method of screening compounds to identify those which stimulate or which inhibit the function of the polypeptide. In general, agonists or antagonists may be employed for therapeutic and prophylactic purposes for such Diseases as hereinbefore mentioned. Compounds may be identified from a variety of sources, for example, cells, cell-free preparations, chemical libraries, and natural product mixtures. Such agonists, antagonists or inhibitors so-identified may be natural or modified substrates, ligands, receptors, enzymes, etc., as the case may be, of the polypeptide; or may be structural or functional mimetics thereof (see Coligan et al., Current Protocols in Immunology 1(2):Chapter 5 (1991)).

The screening method may simply measure the binding of a candidate compound to the polypeptide, or to cells or membranes bearing the polypeptide, or a fusion protein thereof by means of a label directly or indirectly associated with the candidate compound. Alternatively, the screening method may involve competition with a labeled competitor. Further, these screening methods may test whether the candidate compound results in a signal generated by activation or inhibition of the polypeptide, using detection systems appropriate to the cells bearing the polypeptide. Inhibitors of activation are generally assayed in the presence of a known agonist and the effect on activation by the agonist by the presence of the candidate compound is observed. Constitutively active polpypeptides may be employed in screening methods for inverse agonists or inhibitors, in the absence of an agonist or inhibitor, by testing whether the candidate compound results in inhibition of activation of the polypeptide. Further, the screening methods may simply comprise the steps of mixing a candidate compound with a solution containing a polypeptide of the present invention, to form a mixture, measuring SAF-3 activity in the mixture, and comparing the SAF-3 activity of the mixture to a standard. Fusion proteins, such as those made from Fc portion and SAF-3 polypeptide, as hereinbefore described, can also be used for high-throughput screening assays to identify antagonists for the polypeptide of the present invention (see Bennett et al. (1995) J Mol Recognition 8:52-58; and Johanson et al. (1995) J Biol Chem, 270(16):9459-9471).

The polynucleotides, polypeptides and antibodies to the polypeptide of the present invention may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agents which may inhibit or enhance the production of polypeptide (also called antagonist or agonist, respectively) from suitably manipulated cells or tissues.

The polypeptide may be used to identify membrane bound or soluble receptors, if any, through standard receptor binding techniques known in the art. These include, but are not limited to, ligand binding and crosslinking assays in which the polypeptide is labeled with a radioactive isotope (for instance, ¹²⁵I), chemically modified (for instance, biotinylated), or fused to a peptide sequence suitable for detection or purification, and incubated with a source of the putative receptor (cells, cell membranes, cell supernatants, tissue extracts, bodily fluids). Other methods include biophysical techniques such as surface plasmon resonance and spectroscopy. These screening methods may also be used to identify agonists and antagonists of the polypeptide which compete with the binding of the polypeptide to its receptors, if any. Standard methods for conducting such assays are well understood in the art.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypetide of the present invention but do not elicit a response, so that the activity of the polypeptide is prevented.

Thus, in another aspect, the present invention relates to a screening kit for identifying agonists, antagonists, ligands, receptors, substrates, enzymes, etc. for polypeptides of the present invention; or compounds which decrease or enhance the production of such polypeptides, which comprises:

(a) a polypeptide of the present invention;

(b) a recombinant cell expressing a polypeptide of the present invention;

(c) a cell membrane expressing a polypeptide of the present invention; or

(d) antibody to a polypeptide of the present invention;

which polypeptide is preferably that of SEQ ID NO:2.

It will be appreciated that in any such kit, (a), (b), (c) or (d) may comprise a substantial component.

It will be readily appreciated by the skilled artisan that a polypeptide of the present invention may also be used in a method for the structure-based design of an agonist, antagonist or inhibitor of the polypeptide, by:

(a) determining in the first instance the three-dimensional structure of the polypeptide;

(b) deducing the three-dimensional structure for the likely reactive or binding site(s) of an agonist, antagonist or inhibitor;

(c) synthesing candidate compounds that are predicted to bind to or react with the deduced binding or reactive site; and

(d) testing whether the candidate compounds are indeed agonists, antagonists or inhibitors.

It will be further appreciated that this will normally be an interative process.

In a further aspect, the present invention provides methods of treating abnormal conditions such as, for instance, cancer, inflammation, autoimmunity, allergy, asthma, rheumatoid arthritis, CNS inflammation, cererbellar degeneration, Alzheimer's disease, Parkinson's disease, multiple sclerosis, amyotrophic lateral sclerosis, head injury damage, and other neurological abnormalities, septic shock, sepsis, stroke, osteoporosis, osteoarthritis, ischemia reperfusion injury, cardiovascular disease, kidney disease, liver disease, ischemic injury, myocardial infarction, hypotension, hypertension, AIDS, myelodysplastic syndromes and other hematologic abnormalities, aplastic anemia, male pattern baldness, and bacterial, fungal, protozoan and viral infections, related to either an excess of, or an under-expression of, SAF-3 polypeptide activity.

If the activity of the polypeptide is in excess, several approaches are available. One approach comprises administering to a subject in need thereof an inhibitor compound (antagonist) as hereinabove described, optionally in combination with a pharmaceutically acceptable carrier, in an amount effective to inhibit the function of the polypeptide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide may be administered. Typical examples of such competitors include fragments of the SAF-3 polypeptide.

In still another approach, expression of the gene encoding endogenous SAF-3 polypeptide can be inhibited using expression blocking techniques. Known such techniques involve the use of antisense sequences, either internally generated or separately administered (see, for example, O'Connor (1991) J Neurochem 56:560 in Oligodeoxynucleotides as Antisense Inhibitors of Gene Expression, CRC Press, Boca Raton, Fla. (1988)). Alternatively, oligonucleotides which form triple helices with the gene can be supplied (see, for example, Lee et al. (1979) Nucleic Acids Res 6:3073; Cooney et al. (1988) Science 241:456; Dervan et al. (1991) Science 251:1360). These oligomers can be administered per se or the relevant oligomers can be expressed in vivo.

For treating abnormal conditions related to an under-expression of SAF-3 and its activity, several approaches are also available. One approach comprises administering to a subject a therapeutically effective amount of a compound which activates a polypeptide of the present invention, i.e., an agonist as described above, in combination with a pharmaceutically acceptable carrier, to thereby alleviate the abnormal condition. Alternatively, gene therapy may be employed to effect the endogenous production of SAF-3 by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

In a further aspect, the present invention provides for pharmaceutical compositions comprising a therapeutically effective amount of a polypeptide, such as the soluble form of a polypeptide of the present invention, agonist/antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Polypeptides used in treatment can also be generated endogenously in the subject, in treatment modalities often referred to as “gene therapy” as described above. Thus, for example, cells from a subject may be engineered with a polynucleotide, such as a DNA or RNA, to encode a polypeptide ex vivo, and for example, by the use of a retroviral plasmid vector. The cells are then introduced into the subject.

Polynucleotide and polypeptide sequences form a valuable information resource with which to identify further sequences of similar homology. This is most easily facilitated by storing the sequence in a computer readable medium and then using the stored data to search a sequence database using well known searching tools, such as GCC. Accordingly, in a further aspect, the present invention provides for a computer readable medium having stored thereon a polynucleotide comprising the sequence of SEQ ID NO:1 and/or a polypeptide sequence encoded thereby.

The polypeptides of the invention or their fragments or analogs thereof, or cells expressing them, can also be used as immunogens to produce antibodies immunospecific for polypeptides of the present invention. The term “immunospecific” means that the antibodies have substantially greater affinity for the polypeptides of the invention than their affinity for other related polypeptides in the prior art.

Antibodies generated against polypeptides of the present invention may be obtained by administering the polypeptides or epitope-bearing fragments, analogs or cells to an animal, preferably a non-human animal, using routine protocols. For preparation of monoclonal antibodies (“mAbs”), any technique which provides antibodies produced by continuous cell line cultures can be used. Examples include the hybridoma technique (Kohler, G. and Milstein, C. (1975) Nature 256:495-497), the trioma technique, the human B-cell hybridoma technique (Kozbor et al. (1983) Immunology Today 4:72) and the EBV-hybridoma technique (Cole et al. MONOCLONAL ANTIBODIES AND CANCER THERAPY, pp. 77-96, Alan R. Liss, Inc., 1985).

Techniques for the production of single chain antibodies, such as those described in U.S. Pat. No. 4,946,778, can also be adapted to produce single chain antibodies to polypeptides of this invention. Also, transgenic mice, or other organisms, including other mammals, may be used to express humanized antibodies.

For use in constructing the antibodies, altered antibodies and fragments of this invention, a non-human species such as bovine, ovine, monkey, chicken, rodent (e.g., murine and rat) may be employed to generate a desirable immunoglobulin upon presentment with human SAF-3 or a peptide epitope therefrom. Conventional hybridoma techniques are employed to provide a hybridoma cell line secreting a non-human mAb to SAF-3. Such hybridomas are then screened for binding activity as described in the Examples section. Alternatively, fully human mAbs can be generated by techniques known to those skilled in the art.

Exemplary mAbs of the present invention are mAbs 12B1, 2H10, 2G4, 7D9, 13H5, 16F2, 13D5, 16D3 and 12E7, murine antibodies which can be used for the development of chimeric or humanized molecules. These mAbs are characterized by specific binding activity on human SAF-3.

The present invention also includes the use of Fab fragments or F(ab′)₂ fragments derived from mAbs directed against SAF-3 as bivalent fragments. These fragments are useful as agents having binding activity to SAF-3. A Fab fragment contains the entire light chain and amino terminal portion of the heavy chain. An F(ab′)₂ fragment is the fragment formed by two Fab fragments bound by disulfide bonds. The instant antibodies provide sources of Fab fragments and F(ab′)₂ fragments which can be obtained by conventional means, e.g., cleavage of the mAb with the appropriate proteolytic enzymes, papain and/or pepsin, or by recombinant methods. These Fab and F(ab′)₂ fragments are useful themselves as therapeutic, prophylactic or diagnostic agents, and as donors of sequences including the variable regions and CDR sequences useful in the formation of recombinant or humanized antibodies as described herein.

The Fab and F(ab′)₂ fragments can be constructed via a combinatorial phage library (see, e.g., Winter et al. (1994) Ann. Rev. Immunol. 12:433) or via immunoglobulin chain shuffling (see, e.g., Marks et al. (1992) Bio/Technology 10:779), wherein the Fd or V_{H} immunoglobulin from a selected antibody is allowed to associate with a repertoire of light chain immunoglobulins, V_{L} (or V_{K}), to form novel Fabs. Conversely, the light chain immunoglobulin from a selected antibody may be allowed to associate with a repertoire of heavy chain immunoglobulins, V_{H} (or Fd), to form novel Fabs. Anti-SAF-3 mAbs can be obtained by allowing the Fd of said mAbs to associate with a repertoire of light chain immunoglobulins. Hence, one is able to recover Fabs with unique sequences (nucleotide and amino acid) from the chain shuffling technique.

The mAbs of the instant invention may contribute sequences, such as variable heavy and/or light chain peptide sequences, framework sequences, CDR sequences, functional fragments, and analogs thereof, and the nucleic acid sequences encoding them, useful in designing and obtaining various altered antibodies which are characterized by the antigen binding specificity of the donor antibody.

Nucleic acid sequences of this invention, or fragments thereof, encoding the variable light chain and heavy chain peptide sequences, are also useful for mutagenic introduction of specific changes within the nucleic acid sequences encoding the CDRs or framework regions, and for incorporation of the resulting modified or fusion nucleic acid sequence into a plasmid for expression. For example, silent substitutions in the nucleotide sequence of the framework and CDR-encoding regions can be used to create restriction enzyme sites which facilitate insertion of mutagenized CDR and/or framework regions. These CDR-encoding regions can be used in the construction of the humanized antibodies of the invention.

The nucleic and amino acid sequences of the heavy chain variable region of mAb 12B1 is set forth in SEQ ID NOs:5 and 6, respectively. The CDR amino acid sequences from this region are set forth in SEQ ID NOs:9, 10 and 11.

The nucleic and amino acid sequences of the light chain variable region of mAb 12B1 set forth in SEQ ID NO:7 and 8, respectively. The CDR amino acid sequences from this region are set forth in SEQ ID NOs:12, 13 and 14.

Taking into account the degeneracy of the genetic code, various coding sequences may be constructed which encode the variable heavy and light chain amino acid sequences and CDR sequences of the invention as well as functional fragments and analogs thereof which share the antigen specificity of the donor antibody. The isolated nucleic acid sequences of this invention, or fragments thereof, encoding the variable chain peptide sequences or CDRs can be used to produce altered antibodies, e.g., chimeric or humanized antibodies or other engineered antibodies of this invention when operatively combined with a second immunoglobulin partner.

It should be noted that in addition to isolated nucleic acid sequences encoding portions of the altered antibody and antibodies described herein, other such nucleic acid sequences are encompassed by the present invention, such as those complementary to the native CDR-encoding sequences or complementary to the modified human framework regions surrounding the CDR-encoding regions. Useful DNA sequences include those sequences which hybridize under stringent hybridization conditions to the DNA sequences. See, T. Maniatis et al., Molecular Cloning: A Laboratory Manual, Cold Spring Harbor Laboratory (1982), pp. 387-389. Preferred stringent hybridization conditions include overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C. Preferably, these hybridizing DNA sequences are at least about 18 nucleotides in length, i.e., about the size of a CDR.

Altered immunoglobulin molecules can encode altered antibodies which include engineered antibodies such as chimeric antibodies and humanized antibodies. A desired altered immunoglobulin coding region contains CDR-encoding regions that encode peptides having the antigen specificity of an anti-SAF-3 antibody, preferably a high-affinity antibody such as provided by the present invention, inserted into a first immunoglobulin partner such as a human framework or human immunoglobulin variable region.

Preferably, the first immunoglobulin partner is operatively linked to a second immunoglobulin partner. The second immunoglobulin partner is defined above, and may include a sequence encoding a second antibody region of interest, for example an Fe region. Second immunoglobulin partners may also include sequences encoding another immunoglobulin to which the light or heavy chain constant region is fused in frame or by means of a linker sequence. Engineered antibodies directed against functional fragments or analogs of human SAF-3 may be designed to elicit enhanced binding with the same antibody.

The second immunoglobulin partner may also be associated with effector agents as defined above, including non-protein carrier molecules, to which the second immunoglobulin partner may be operatively linked by conventional means.

Fusion or linkage between the second immunoglobulin partners, e.g., antibody sequences, and the effector agent, may be by any suitable means, e.g., by conventional covalent or ionic bonds, protein fusions, or hetero-bifunctional cross-linkers, e.g., carbodiimide, glutaraldehyde and the like. Such techniques are known in the art and are described in conventional chemistry and biochemistry texts.

Additionally, conventional linker sequences which simply provide for a desired amount of space between the second immunoglobulin partner and the effector agent may also be constructed into the altered immunoglobulin coding region. The design of such linkers is well known to those of skill in the art.

In addition, signal sequences for the molecules of the invention may be modified by techniques known to those skilled in the art to enhance expression and intra- and intercellular trafficing.

A preferred altered antibody contains a variable heavy and/or light chain peptide or protein sequence having the antigen specificity of mAb 12B1, e.g., the V_{H} and V_{K} chains. Still another desirable altered antibody of this invention is characterized by the amino acid sequence containing at least one, and preferably all of the CDRs of the variable region of the heavy and/or light chains of the murine antibody molecule 12B1 with the remaining sequences being derived from a human source, or a functional fragment or analog thereof.

In a further embodiment, the altered antibody of the invention may have attached to it an additional agent. For example, recombinant DNA technology may be used to produce an altered antibody of the invention in which the Fe fragment or CH2 CH3 domain of a complete antibody molecule has been replaced by an enzyme or other detectable molecule, i.e., a polypeptide effector or reporter molecule. Other additional agents include toxins, antiproliferative drugs and radionuclides.

The second immunoglobulin partner may also be operatively linked to a non-immunoglobulin peptide, protein or fragment thereof heterologous to the CDR-containing sequence having antigen specificity to human SAF-3. The resulting protein may exhibit both antigen specificity and characteristics of the non-immunoglobulin upon expression. That fusion partner characteristic may be, for example, a functional characteristic such as another binding or receptor domain or a therapeutic characteristic if the fusion partner is itself a therapeutic protein or additional antigenic characteristics.

Another desirable protein of this invention may comprise a complete antibody molecule, having full length heavy and light chains or any discrete fragment thereof, such as the Fab or F(ab′)₂ fragments, a heavy chain dimer or any minimal recombinant fragments thereof such as an Fv or a single-chain antibody (SCA) or any other molecule with the same specificity as the selected donor monoclonal antibody. Such protein may be used in the form of an altered antibody or may be used in its unfused form.

Whenever the second immunoglobulin partner is derived from an antibody different from the donor antibody, e.g., any isotype or class of immunoglobulin framework or constant regions, an engineered antibody results. Engineered antibodies can comprise immunoglobulin constant regions and variable framework regions from one source, e.g., the acceptor antibody, and one or more (preferably all) CDRs from the donor antibody. In addition, alterations, e.g., deletions, substitutions, or additions, of the acceptor mAb light and/or heavy variable domain framework region at the nucleic acid or amino acid levels, or the donor CDR regions may be made in order to retain donor antibody antigen binding specificity.

Such engineered antibodies are designed to employ one (or both) of the variable heavy and/or light chains of an anti-SAF-3 mAb (optionally modified as described) or one or more of the heavy or light chain CDRs. The engineered antibodies of the invention exhibit binding activity.

Such engineered antibodies may include a humanized antibody containing the framework regions of a selected human immunoglobulin or subtype or a chimeric antibody containing the human heavy and light chain constant regions fused to the anti-SAF-3 mAb functional fragments. A suitable human (or other animal) acceptor antibody may be one selected from a conventional database, e.g., the KABAT® database, Los Alamos database, and Swiss Protein database, by homology to the nucleotide and amino acid sequences of the donor antibody. A human antibody characterized by a homology to the V region frameworks of the donor antibody or V region subfamily consensus sequences (on an amino acid basis) may be suitable to provide a heavy chain variable framework region for insertion of the donor CDRs. A suitable acceptor antibody capable of donating light chain variable framework regions may be selected in a similar manner. It should be noted that the acceptor antibody heavy and light chains are not required to originate from the same acceptor antibody.

Preferably, the heterologous framework and constant regions are selected from human immunoglobulin classes and isotypes, such as IgG (subtypes 1 through 4), IgM, IgA, and IgE. IgG1, k and IgG4, k are preferred. Particularly preferred is IgG 4, k. Most particularly preferred is the IgG4 subtype variant containing the mutations S228P and L235E (PE mutation) in the heavy chain constant region which results in reduced effector function. This IgG4 subtype variant is known herein as IgG4PE. See U.S. Pat. Nos. 5,624,821 and 5,648,260.

The acceptor antibody need not comprise only human immunoglobulin protein sequences. For instance, a gene may be constructed in which a DNA sequence encoding part of a human immunoglobulin chain is fused to a DNA sequence encoding a non-immunoglobulin amino acid sequence such as a polypeptide effector or reporter molecule.

A particularly preferred humanized antibody contains CDRs of mAb 12B1 inserted into the framework regions of a selected human antibody sequence. For humanized antibodies, one, two or preferably three CDRs from mAb 12B1 heavy chain and/or light chain variable regions are inserted into the framework regions of the selected human antibody sequence, replacing the native CDRs of the human antibody.

Preferably, in a humanized antibody, the variable domains in both human heavy and light chains have been engineered by one or more CDR replacements. It is possible to use all six CDRs, or various combinations of less than the six CDRs. Preferably all six CDRs are replaced. It is possible to replace the CDRs only in the human heavy chain, using as light chain the unmodified light chain from the human acceptor antibody. Still alternatively, a compatible light chain may be selected from another human antibody by recourse to conventional antibody databases. The remainder of the engineered antibody may be derived from any suitable acceptor human immunoglobulin.

The engineered humanized antibody thus preferably has the structure of a natural human antibody or a fragment thereof, and possesses the combination of properties required for effective therapeutic use such as the treatment of allergic rhinitis, allergies, asthma, eczema, or diseases such as lymphoma, leukemia, or systemic mastocytosis.

It will be understood by those skilled in the art that an engineered antibody may be further modified by changes in variable domain amino acids without necessarily affecting the specificity and high affinity of the donor antibody (i.e., an analog). It is anticipated that heavy and light chain amino acids may be substituted by other amino acids either in the variable domain frameworks or CDRs or both. These substitutions could be supplied by the donor antibody or consensus sequences from a particular subgroup.

In addition, the constant region may be altered to enhance or decrease selective properties of the molecules of this invention. For example, dimerization, binding to Fc receptors, or the ability to bind and activate complement (see, e.g., Angal et al. (1993) Mol. Immunol. 30:105; Xu et al. (1994) J. Biol. Chem. 269: 3469; European Patent Publication No. EP 0 307 434 B1).

An altered antibody which is a chimeric antibody differs from the humanized antibodies described above by providing the entire non-human donor antibody heavy chain and light chain variable regions, including framework regions, in association with human immunoglobulin constant regions for both chains. It is anticipated that chimeric antibodies which retain additional non-human sequence relative to humanized antibodies of this invention may be useful for treating cancer, inflammation, autoimmunity, allergy, asthma, rheumatoid arthritis, CNS inflammation, multiple sclerosis, AIDS, and bacterial, fungal, protozoan and viral infections.

Preferably, the variable light and/or heavy chain sequences and the CDRs of mAb 12B1 or other suitable donor mAbs and their encoding nucleic acid sequences, are utilized in the construction of altered antibodies, preferably humanized antibodies, of this invention, by the following process. The same or similar techniques may also be employed to generate other embodiments of this invention.

A hybridoma producing a selected donor mAb, e.g., the murine antibody 12B1, is conventionally cloned and the DNA of its heavy and light chain variable regions obtained by techniques known to one of skill in the art, e.g., the techniques described in Sambrook et al., Molecular Cloning: A Laboratory Manual, 2nd edition, Cold Spring Harbor Laboratory (1989). The variable heavy and light regions containing at least the CDR-encoding regions and those portions of the acceptor mAb light and/or heavy variable domain framework regions required in order to retain donor mAb binding specificity, as well as the remaining immunoglobulin-derived parts of the antibody chain derived from a human immunoglobulin, are obtained using polynucleotide primers and reverse transcriptase. The CDR-encoding regions are identified using a known database and by comparison to other antibodies.

A mouse/human chimeric antibody may then be prepared and assayed for binding ability. Such a chimeric antibody contains the entire non-human donor antibody V_{H} and V_{L} regions, in association with human Ig constant regions for both chains.

Homologous framework regions of a heavy chain variable region from a human antibody are identified using computerized databases, e.g., KABAT®, and a human antibody characterized by homology to the V region frameworks of the donor antibody or V region subfamily consensus sequences (on an amino acid basis) to mAb 12B1 is selected as the acceptor antibody. The sequences of synthetic heavy chain variable regions containing the CDR-encoding regions within the human antibody frameworks are designed with optional nucleotide replacements in the framework regions to incorporate restriction sites. This designed sequence is then synthesized using long synthetic oligomers. Alternatively, the designed sequence can be synthesized by overlapping oligonucleotides, amplified by polymerase chain reaction (PCR), and corrected for errors. A suitable light chain variable framework region can be designed in a similar manner.

A humanized antibody may be derived from the chimeric antibody, or preferably, made synthetically by inserting the donor mAb CDR-encoding regions from the heavy and light chains appropriately within the selected heavy and light chain framework. Alternatively, a humanized antibody of the invention may be prepared using standard mutagenesis techniques. Thus, the resulting humanized antibody contains human framework regions and donor mAb CDR-encoding regions. There may be subsequent manipulation of framework residues. The resulting humanized antibody can be expressed in recombinant host cells, e.g., COS, CHO or myeloma cells.

A conventional expression vector or recombinant plasmid is produced by placing these coding sequences for the altered antibody in operative association with conventional regulatory control sequences capable of controlling the replication and expression in, and/or secretion from, a host cell. Regulatory sequences include promoter sequences, e.g., CMV or Rous Sarcoma virus promoter, and signal sequences, which can be derived from other known antibodies. Similarly, a second expression vector can be produced having a DNA sequence which encodes a complementary antibody light or heavy chain. Preferably, this second expression vector is identical to the first except with respect to the coding sequences and selectable markers, in order to ensure, as much as possible, that each polypeptide chain is functionally expressed. Alternatively, the heavy and light chain coding sequences for the altered antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The humanized antibody which includes the association of both the recombinant heavy chain and/or light chain is screened from culture by an appropriate assay such as ELISA or RIA. Similar conventional techniques may be employed to construct other altered antibodies and molecules of this invention.

Suitable vectors for the cloning and subcloning steps employed in the methods and construction of the compositions of this invention may be selected by one of skill in the art. For example, the pUC series of cloning vectors, such as pUC19, which is commercially available from vendors such as Amersham or Pharmacia, may be used. Additionally, any vector which is capable of replicating readily, has an abundance of cloning sites and selectable genes (e.g. antibiotic resistance), and is easily manipulated may be used for cloning. Thus, the selection of the cloning vector is not a limiting factor in this invention.

Similarly, the vectors employed for expression of the engineered antibodies according to this invention may be selected by one of skill in the art from any conventional vector. The vectors also contain selected regulatory sequences (such as CMV or Rous Sarcoma virus promoters) which direct the replication and expression of heterologous DNA sequences in selected host cells. These vectors contain the above-described DNA sequences which code for the engineered antibody or altered immunoglobulin coding region. In addition, the vectors may incorporate the selected immunoglobulin sequences modified by the insertion of desirable restriction sites for ready manipulation.

The expression vectors may also be characterized by genes suitable for amplifying expression of the heterologous DNA sequences, e.g., the mammalian dihydrofolate reductase gene (DHFR). Other preferable vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g., replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast and fungal expression may also be selected for this purpose.

The present invention also encompasses a cell line transfected with a recombinant plasmid containing the coding sequences of the engineered antibodies or altered immunoglobulin molecules thereof. Host cells useful for the cloning and other manipulations of these cloning vectors are also conventional. However, most desirably, cells from various strains of E. coli are used for replication of the cloning vectors and other steps in the construction of altered antibodies of this invention.

Suitable host cells or cell lines for the expression of the engineered antibody or altered antibody of the invention are preferably mammalian cells such as CHO, COS, a fibroblast cell (e.g., 3T3) and myeloid cells, and more preferably a CHO or a myeloid cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., supra.

Bacterial cells may prove useful as host cells suitable for the expression of the recombinant Fabs of the present invention (see, e.g., Plückthun, A. (1992) Immunol. Rev. 130:151-188). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host. For example, various strains of E. coli used for expression are well-known as host cells in the field of biotechnology. Various strains of B. subtilis, Streptomyces, other bacilli and the like may also be employed.

Where desired, strains of yeast cells known to those skilled in the art are also available as host cells, as well as insect cells, e.g. Drosophila and Lepidoptera, and viral expression systems. See, e.g. Miller et al., Genetic Engineering, 8, 277-298, Plenum Press (1986) and references cited therein.

The general methods by which the vectors of the invention may be constructed, the transfection methods required to produce the host cells of the invention, and culture methods necessary to produce the altered antibody of the invention from such host cell are all conventional techniques. Likewise, once produced, the altered antibodies of the invention may be purified from the cell culture contents according to standard procedures of the art, including ammonium sulfate precipitation, affinity columns, column chromatography, gel electrophoresis and the like. Such techniques are within the skill of the art and do not limit this invention.

Yet another method of expression of the humanized antibodies may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

Once expressed by the desired method, the engineered antibody is then examined for in vitro activity by use of an appropriate assay.

Following the procedures described for humanized antibodies prepared from the instant mAbs, one of skill in the art may also construct humanized antibodies from other donor antibodies, variable region sequences and CDR peptides described herein. Engineered antibodies can be produced with variable region frameworks potentially recognized as “self” by recipients of the engineered antibody. Modifications to the variable region frameworks can be implemented to effect increases in antigen binding and antagonist activity without appreciable increased immunogenicity for the recipient. Such engineered antibodies may effectively treat a human for ischemic diseases such as myocardial infarction or cerebral stroke or treatment of vascular insufficiency diseases, such as diabetes. Such antibodies may also be useful in the diagnosis of those conditions.

This invention also relates to a method for treating cancer, inflammation, autoimmunity, allergy, asthma, rheumatoid arthritis, CNS inflammation, multiple sclerosis, AIDS, and bacterial, fungal, protozoan and viral infections in a mammal, particularly a human, which comprises administering an effective dose of an anti-SAF-3 monoclonal antibody. The mAb can include one or more of the antibodies or altered antibodies described herein or fragments thereof. Thus, the molecules of the present invention, when in preparations and formulations appropriate for therapeutic use, are highly desirable for persons susceptible to or experiencing cancer, inflammation, autoimmunity, allergy, asthma, rheumatoid arthritis, CNS inflammation, multiple sclerosis, AIDS, and bacterial, fungal, protozoan and viral infections.

The monoclonal antibodies used in the methods of the invention can include one or more of the antibodies or altered antibodies described herein or fragments thereof. Preferably, the anti-SAF-3 antibody used in the methods of the invention has the identifying characteristics of any of mAbs 12B1, 2H10, 2G4, 7D9, 13H5, 16F2, 13D5, 16D3 and 12E7.

The altered antibodies, antibodies and fragments thereof of this invention may also be used in conjunction with other antibodies, particularly human mAbs reactive with other markers (epitopes) responsible for the condition against which the engineered antibody of the invention is directed.

The antibodies of the present invention can be formulated into pharmaceutical compositions and administered in the same manner as described for mature proteins. See, e.g., International Patent Application, Publication No. WO90/02762. Generally, these compositions contain a therapeutically effective amount of an antibody of this invention and an acceptable pharmaceutical carrier. Suitable carriers are well known to those of skill in the art and include, for example, saline. Alternatively, such compositions may include conventional delivery systems into which protein of the invention is incorporated. Optionally, these compositions may contain other active ingredients.

The antibodies of this invention may be administered by any appropriate internal route, and may be repeated as needed, e.g., as frequently as one to three times daily for between 1 day to about three weeks to once per week or once biweekly. Preferably, the antibody is administered less frequently than is the ligand, when it is used therapeutically. The dose and duration of treatment relates to the relative duration of the molecules of the present invention in the human circulation, and can be adjusted by one of skill in the art depending upon the condition being treated and the general health of the patient.

As used herein, the term “pharmaceutical” includes veterinary applications of the invention. The term “therapeutically effective amount” refers to that amount of an antibody, which is useful for alleviating a selected condition. These therapeutic compositions of the invention may be administered to mimic the effect of the normal receptor ligand.

The mode of administration of the antibodies of the invention may be any suitable route which delivers the agent to the host. The altered antibodies, antibodies, engineered antibodies, and fragments thereof, and pharmaceutical compositions of the invention are particularly useful for parenteral administration, i.e., subcutaneously, intramuscularly, intravenously or intranasally.

Antibodies of the invention may be prepared as pharmaceutical compositions containing an effective amount of the engineered (e.g., humanized) antibody of the invention as an active ingredient in a pharmaceutically acceptable carrier. In the compositions of the invention, an aqueous suspension or solution containing the engineered antibody, preferably buffered at physiological pH, in a form ready for injection is preferred. The compositions for parenteral administration will commonly comprise a solution of the engineered antibody of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of an engineered antibody of the invention. Similarly, a pharmaceutical composition of the invention for intravenous infusion could be made up to contain about 250 mL of sterile Ringer's solution, and about 1 mg to about 30 mg and preferably 5 mg to about 25 mg of an engineered antibody of the invention. Actual methods for preparing parenterally administrable compositions are well known or will be apparent to those skilled in the art and are described in more detail in, for example, “Remington's Pharmaceutical Science”, 15th ed., Mack Publishing Company, Easton, Pa.

It is preferred that the antibodies of the invention, when in a pharmaceutical preparation, be present in unit dose forms. The appropriate therapeutically effective dose can be determined readily by those of skill in the art. To effectively treat anemia in a human or other animal, one dose of approximately 0.01 mg to approximately 20 mg per kg body weight of a protein or an antibody of this invention should be administered parenterally, preferably i.v. or i.m. Such dose may, if necessary, be repeated at appropriate time intervals selected as appropriate by a physician during the response period.

Antibodies against SAF-3 polypeptides may also be employed to subcharacterize cell populations during hematopoietic development, as a diagnostic marker to distinguish between different forms of cancer, to purge bone marrow ex vivo of cancer cells expressing SAF-3, as a tool to aid in the ex vivo expansion (proliferation and/or differentiation) of hematopoietic progenitor cells expressing SAF-3, as a stimulus in vivo for stem cell mobilization into the periphery, and as an in vivo chemoprotective agent.

Definitions

The following definitions are provided to facilitate understanding of certain terms used frequently hereinbefore.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al. (1990) Meth Enzymol 182:626-646 and Rattan et al. (1992) Ann NY Acad Sci 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity” is a measure of the identity of nucleotide sequences or amino acid sequences. In general, the sequences are aligned so that the highest order match is obtained. “Identity” per se has an art-recognized meaning and can be calculated using published techniques (see, e.g.: COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. and Lipton, D. (1988) SIAM J Applied Math 48:1073). Methods commonly employed to determine identity or similarity between two sequences include, but are not limited to, those disclosed in Guide to Huge Computers, Martin J. Bishop, ed., Academic Press, San Diego, 1994, and Carillo, H. and Lipton, D. (1988) SIAM J Applied Math 48:1073. Methods to determine identity and similarity are codified in computer programs. Preferred computer program methods to determine identity and similarity between two sequences include, but are not limited to, GCG program package (Devereux, J. et al. (1984) Nucleic Acids Research 12(1):387), BLASTP, BLASTN, and FASTA (Atschul, S. F. et al. (1990) J Molec Biol 215:403).

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity(divided by 100) and subtracting that product from said total number of nucleotides in SEQ ID NO:1, or:
nₙ≦xₙ−(xₙ·y),
wherein nₙ is the number of nucleotide alterations, xₙ is the total number of nucleotides in SEQ ID NO:1, and y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity(divided by 100) and then subtracting that product from said total number of amino acids in SEQ ID NO:2, or:
nₐ≦xₐ−(xₐ·y),
wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xₐ.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

“Antibodies” refers to immunoglobulins which can be prepared by conventional hybridoma techniques, phage display combinatorial libraries, immunoglobulin chain shuffling and humanization techniques. Also included are fully human monoclonal antibodies. As used herein, “antibody” also includes “altered antibody” which refers to a protein encoded by an altered immunoglobulin coding region, which may be obtained by expression in a selected host cell. Such altered antibodies are engineered antibodies (e.g., chimeric or humanized antibodies) or antibody fragments lacking all or part of an immunoglobulin constant region, e.g., Fv, Fab, Fab′ or F(ab′)₂ and the like. The terms Fv, Fc, Fd, Fab, Fab′ or F(ab′)₂ are used with their standard meanings. See, e.g., Harlow et al. in “Antibodies A Laboratory Manual”, Cold Spring Harbor Laboratory, (1988).

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest in this invention are derived from donor antibody variable heavy and light chain sequences, and include analogs of the naturally occurring CDRs, which analogs share or retain the same antigen binding specificity and/or antagonist ability as the donor antibody from which they were derived, yet may exhibit increased affinity for the antigen. An exemplary process for obtaining analogs is affinity maturation by means of phage display technology as reviewed by Hoogenboom (1997) Trends in Biotechnology 15:62; Barbas et al. (1996) Trends in Biotechnology 14:230; and Winter et al. (1994) Ann. Rev. Immunol. 12:433 and described by Irving et al. (1996) Immunotechnology 2:127.

“Altered immunoglobulin coding region” refers to a nucleic acid sequence encoding an altered antibody of the invention. When the altered antibody is a complementarity determining region-grafted (CDR-grafted) or humanized antibody, the sequences that encode the CDRs from a non-human immunoglobulin are inserted into a first immunoglobulin partner comprising human variable framework sequences. Optionally, the first immunoglobulin partner is operatively linked to a second immunoglobulin partner.

“First immunoglobulin partner” refers to a nucleic acid sequence encoding a human framework or human immunoglobulin variable region in which the native (or naturally-occurring) CDR-encoding regions are replaced by the CDR-encoding regions of a donor antibody. The human variable region can be an immunoglobulin heavy chain, a light chain (or both chains), an analog or functional fragments thereof. Such CDR regions, located within the variable region of antibodies (immunoglobulins) can be determined by known methods in the art. For example Kabat et al. in “Sequences of Proteins of Immunological Interest”, 4th Ed., U.S. Department of Health and Human Services, National Institutes of Health (1987) disclose rules for locating CDRs. In addition, computer programs are known which are useful for identifying CDR regions/structures.

“Second immunoglobulin partner” refers to another nucleotide sequence encoding a protein or peptide to which the first immunoglobulin partner is fused in frame or by means of an optional conventional linker sequence (i.e., operatively linked). Preferably, it is an immunoglobulin gene. The second immunoglobulin partner may include a nucleic acid sequence encoding the entire constant region for the same (i.e., homologous, where the first and second altered antibodies are derived from the same source) or an additional (i.e., heterologous) antibody of interest. It may be an immunoglobulin heavy chain or light chain (or both chains as part of a single polypeptide). The second immunoglobulin partner is not limited to a particular immunoglobulin class or isotype. In addition, the second immunoglobulin partner may comprise part of an immunoglobulin constant region, such as found in a Fab, or F(ab′)₂ (i.e., a discrete part of an appropriate human constant region or framework region). Such second immunoglobulin partner may also comprise a sequence encoding an integral membrane protein exposed on the outer surface of a host cell, e.g., as part of a phage display library, or a sequence encoding a protein for analytical or diagnostic detection, e.g., horseradish peroxidase, β-galactosidase, etc.

As used herein, an “engineered antibody” describes a type of altered antibody, i.e., a full-length synthetic antibody (e.g., a chimeric or humanized antibody as opposed to an antibody fragment) in which a portion of the light and/or heavy chain variable domains of a selected acceptor antibody are replaced by analogous parts from one or more donor antibodies which have specificity for the selected epitope. For example, such molecules may include antibodies characterized by a humanized heavy chain associated with an unmodified light chain (or chimeric light chain), or vice versa. Engineered antibodies may also be characterized by alteration of the nucleic acid sequences encoding the acceptor antibody light and/or heavy variable domain framework regions in order to retain donor antibody binding specificity. These antibodies can comprise replacement of one or more CDRs (preferably all) from the acceptor antibody with CDRs from a donor antibody described herein.

The term “donor antibody” refers to a monoclonal or recombinant antibody which contributes the nucleic acid sequences of its variable regions, CDRs or other functional fragments or analogs thereof to a first immunoglobulin partner, so as to provide the altered immunoglobulin coding region and resulting expressed altered antibody with the antigenic specificity and neutralizing activity characteristic of the donor antibody. Donor antibodies suitable for use in this invention is a murine monoclonal antibody designated as 2C4.

The term “acceptor antibody” refers to monoclonal or recombinant antibodies heterologous to the donor antibody, which contributes all, or a portion, of the nucleic acid sequences encoding its heavy and/or light chain framework regions and/or its heavy and/or light chain constant regions or V region subfamily consensus sequences to the first immunoglobulin partner. Preferably, a human antibody is the acceptor antibody.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulins. In addition, framework support residues may be altered to preserve binding affinity. See, e.g., Queen et al. (1089) Proc. Natl Acad Sci USA 86:10029; Hodgson et al. (1991) Bio/Technology 9:421). Furthermore, as described herein, additional residues may be altered to preserve the activity of the donor antibody.

A “functional fragment” is a partial heavy or light chain variable sequence (e.g., minor deletions at the amino or carboxy terminus of the immunoglobulin variable region) which shares the same antigen binding specificity as the antibody from which the fragment was derived.

An “analog” is an amino acid sequence modified by at least one amino acid, wherein said modification can be chemical or a substitution or a rearrangement of a few amino acids (i.e., no more than 10) and corresponding nucleic acid sequences, which modification permits the amino acid sequence to retain the biological characteristics, e.g., antigen specificity and high affinity, of the unmodified sequence. Exemplary nucleic acid analogs include silent mutations which can be constructed, via substitutions, to create certain endonuclease restriction sites within or surrounding CDR-encoding regions.

Analogs may also arise as allelic variations. An “allelic variation or modification” is an alteration in the nucleic acid sequence encoding the amino acid or peptide sequences of the invention. Such variations or modifications may be due to degeneracy in the genetic code or may be deliberately engineered to provide desired characteristics. These variations or modifications may or may not result in alterations in any encoded amino acid sequence.

By “sharing the antigen binding specificity” is meant, for example, that although mAb 12B1 may be characterized by a certain level of binding activity, a polypeptide encoding a CDR derived from mAb 12B1 in any appropriate structural environment may have a lower or higher activity. It is expected that CDRs of mAb 12B1 in such environments will nevertheless recognize the same epitope(s) as mAb 12B1.

The phrase “having the identifying characteristics of” as used herein indicates that such antibodies or polypeptides share the same antigen binding specificity as the antibodies exemplified herein, and bind to SAF-3 with a substantially similar affinity as the antibodies exemplified herein as measured by methods well known to those skilled in this art. Thus, functional fragments of the antibodies described herein would have the identifying characteristics of the antibodies from which they are derived. Moreover, antibodies that have the same or analogous CDRs as the antibodies exemplified herein, and thus bind to SAF-3 with a substantially similar affinity as the antibodies exemplified herein, have the same identifying characteristics as the antibodies exemplified herein. Antibodies that share identifying characteristics may be engineered antibodies, chimeric antibodies and humanized antibodies, and functional fragments thereof.

The term “effector agents” refers to non-protein carrier molecules to which the altered antibodies, and/or natural or synthetic light or heavy chains of the donor antibody or other fragments of the donor antibody may be associated by conventional means. Such non-protein carriers can include conventional carriers used in the diagnostic field, e.g., polystyrene or other plastic beads, polysaccharides, e.g., as used in the BIAcore (Pharmacia) system, or other non-protein substances useful in the medical field and safe for administration to humans and animals. Other effector agents may include a macrocycle, for chelating a heavy metal atom or radioisotopes. Such effector agents may also be useful to increase the half-life of the altered antibodies, e.g., polyethylene glycol.

As used herein, the term “treating” and derivatives thereof means prophylactic, palliative or therapeutic therapy.

The present invention may be embodied in other specific forms, without departing from the spirit or essential attributes thereof, and, accordingly, reference should be made to the appended claims, rather than to the foregoing specification or following examples, as indicating the scope of the invention.

All publications including, but not limited to, patents and patent applications, cited in this specification or to which this patent application claims priority, are herein incorporated by reference as if each individual publication were specifically and individually indicated to be incorporated by reference herein as though fully set forth.

### EXAMPLES

The present invention will now be described with reference to the following specific, non-limiting examples.

### Example 1

Generation of Monoclonal Antibodies

A DNA fragment encoding the polypeptide set forth in SEQ ID NO:2 was subcloned into the mammalian expression vector pCDN (see Aiyar et al. (1994) Mol. Cell Biochem. 131:75-86) using PCR. The sequence of the insert was confirmed before being transfected into HEK293 cells using Ca⁺⁺PO₄. Clones were selected in 500 ug/ml G418 and evaluated for expression using Northern blot analysis followed by FACS analysis. The extracellular domain of SAF-3 was subcloned by PCR and inserted in frame with a Factor Xa cleavage site and the Fc portion of human IgG4 or human IgG1. The sequence was confirmed before the vectors were electroporated into CHOEA1 cells. Stably expressing clones were selected, expanded, evaluated for Fc expression and scaled up. SAF-3/Fc fusion was purified from supernatant using Protein A Sepherose and an aliquot was cleaved with Factor Xa to generate the SAF-3 used for antibody generation.

Mice were immunized with SAF-3 (25 ug) in Freund's complete adjuvant and then received two booster injections (25 ug) at 2 and 4 weeks. On the basis of a good serum antibody titer to SAF-3, one mouse received a further immunization of 20 ug of SAF-3 i.v. in PBS. The spleen was harvested four days later and fused with myeloma cells according to the method described in Zola (Zola, H. (1987) Monoclonal antibodies: A manual of techniques. CRC Press, Boca Raton, Fla.).

Positive hybridomas were tested for binding in 96 well microtiter plates coated with SAF-3/Fc at 0.5 ug/mL and detected with europium conjugated anti-mouse IgG. Specifically, 96-well plates were coated with SAF-3/Fc (100 uL/well in PBS) by incubation overnight at 4° C. The solution was then aspirated and non-specific binding sites were blocked with 250 μL/well of 1% bovine serum albumin (BSA) in TBS buffer (50 mM Tris, 150 mM NaCl, 0.02% Kathon, pH 7.4) for 5-60 minutes at RT. Following this and each of the following steps, the plate was washed 4 times in wash buffer (10 mM Tris, 150 mM NaCl, 0.05% Tween 20, 0.02% Kathon, pH 7.4). To each well, 50 μL hybridoma medium and 50 μl assay buffer (0.5% BSA, 0.05% bovine gamma globulin, 0.01% Tween 40, 20 μM diethylenetriaminepentaacetic acid in TBS buffer) was added and incubated for 60 minutes at RT in a shaker-incubator. To each well was then added 100 μL 0.5 μg/mL Eu3+ labeled anti-mouse antibody in assay buffer. Finally, 200 μL/well of enhancer (Wallac, Tuku, Finland) was added and incubated for 5 minutes at RT, and the time-resolved fluorescence measured. Positives were rescreened by immunoassay and BIAcore and then cloned by the limiting dilution method.

Monoclonal antibodies were purified by ProsepA (Bio Processing, Consett, UK) chromatography, respectively, using the manufacturer's instructions. Monoclonal antibodies were >95% pure by SDS-PAGE.

### Example 2

Characterization of Monoclonal Antibodies

Positive hybridomas that were identified by immunoassay were confirmed by flow cytometry using the 293 transfected stable cell lines. Monoclonal antibodies disclosed herein were isotyped using commercially available reagents (Pharmingen, San Diego, Calif.). Table 1 lists the antibodies and their isotypes.

TABLE 1

| Antibody | Isotype |
|---|---|
| 12B1 | IgG2a, k |
| 2H10 | IgG2b, k |
| 2G4 | IgG2b, k |
| 7D9 | IgG2b, k |
| 13H5 | IgG2b, k |
| 16F2 | IgG1, k |
| 13D5 | IgG1, k |
| 16D3 | IgG1, k |
| 12E7 | IgG1, k |

No evidence of crossreactivity by immunoassay at the concentrations tested (max. displacement with SAF-1 or -2 was 5% compared to displacement of 10-90% with SAF-3). By BIACore analysis, 13H5 crossreacted with SAF-2; no other crossreactions were observed.

Epitope mapping of the clones is detailed in the table below:

TABLE 2

| | Antibody recognizing | Antibody recognizing |
|---|---|---|
| Antibody | a distinct epitope | an overlapping epitope |
| 12B1 | 2H10, 2G4, 7D9, 13H5, 13D5 | 16D3 |
| 16F2 | 2H10, 2G4, 7D9, 13H5, 12E7, 13D5, | |
| | 16D3 | |
| 2H10 | 12B1, 16F2, 12E7, 16D3 | |
| 2G4 | 12B1, 16F2, 13H5, 12E7, 16D3 | |
| 7D9 | 12B1, 16F2, 13H5, 12E7, 16D3 | |
| 13H5 | 12B1, 16F2, 2G4, 7D9, 12E7, 13D5 | |
| 12E7 | 16F2, 2H10, 2G4, 7D9, 13H5, 13D5 | 16D3 |
| 13D5 | 12B1, 16F2, 13H5, 12E7, 16D3 | |
| 16D3 | 16F2, 2H10, 2G4, 7D9, 13D5 | 12B1, 12E7 |

12B1 and 16F2 probably recognize distinct epitopes. An additional approach to define the area of binding for each mAb is the expression of two Ig domain (1 & 3) verses the three Ig domain (1, 2 & 3) SAF-3. Transient expression on 293 cells has demonstrated that binding of 13H5 and 16D3 are abrogated when the two Ig domain SAF-3 is expressed. Binding of 12E7 is altered but not completely eliminated. These data indicate that the epitope of 13H5 and 16D3 is: 1) within the second Ig domain; 2) at the junction of the first and second Ig domains; or that there is a conformational change of the binding site of the antibodies due to the elimination of the second Ig domain.

The mAbs for SAF-3 were used to evaluate SAF-3 expression on leukocytes from whole blood, bone marrow and cultured cells. Expression of SAF-3 has been observed on monocytes, NK cells and dendritic cells cultured from CD 14+ and CD34+ derived cells (Table 3).

TABLE 3

Mean Fluorescent Intensity

| | Isotype | |
|---|---|---|
| Cell Type | Control | Anti-SAF-3 |
| CD14+ derived DCs | 4.9 | 39.7 |
| CD34+ derived DCs | 7.6 | 69.6 |
| CD14+ monocytes | 5.7 | 20.1 |
| NK cells | 4.1 | 30.4 |

SAF-3/Fc fusion was tested for binding to lymphocytes, neutrophils and eosinophils. Using flow cytometry, there was strong binding of the Fc fusion to lymphocytes but not neutrophils or eosinophils. Using two color immunofluoresence, the binding of SAF-3/Fc was on all CD3+ cells and approximately half of the CD19+ and CD20+ cells. It appears that the sialic acids specific for SAF-3 binding are expressed by T cells and some B cells.

Modulation of Immune Response

Initiation and maintenance of an immune response is mediated primarily by dendritic cells (DCs). DCs traffic throughout the body gathering antigens and bringing them to lymph nodes where they then present antigen to T and B cells. The ability to inhibit or enhance DC function would be of great benefit in autoimmune disease or cancer immunotherapy, respectively. The autoimmune diseases with the greatest unmet medical need and commercial opportunity are rheumatoid arthritis, psoriasis, multiple sclerosis, inflammatory bowel disease and systemic lupus erythematosus.

Dendritic cells are “professional” antigen presenting cells (APC). Their function is to capture antigen (i.e. from sites of infection or tumor), process the antigen and move from the periphery to the secondary lymphoid organs (lymph nodes), and present the antigen to T cells. They provide the co-stimulatory molecules and cytokines necessary for T cell proliferation and effector function. The activated T cells will either stay in the node and provide help (T helper cells) to B cells or other T cells, or leave the lymph nodes and move to the site of infection or tumor and exert their biologic effect (cytotoxicity or cytokine production).

Modulation of SAF-3 function on DCs would affect autoimmune disease, by downregulation of the immune response, or enhance the immune response to malignant tumors. Autoimmunity results when the immune system becomes dysregulated and begins attacking an organ system: the lining of the joints in rheumatoid arthritis or the white matter of the brain and spinal cord in multiple sclerosis. Conversely, in many patients with tumors, the immune system fails to recognize the tumor resulting in uncontrolled growth. Agents which directly effect the underlying immune response would provide novel mechanisms for treatment of these diseases.

Monoclonal antibodies to SAF-3 that inhibit DC stimulation of T cells could be used for treatment of autoimmune disorders, primarily rheumatoid arthritis. These “antagonist” SAF-3 mAbs would be expected to be disease modifying: interruption of continued T cell stimulation by DCs may allow re-establishment of the proper regulation mechanisms and cause disease suppression. Current therapies have a general immunosuppressive effect (methotrexate, corticosteroids).

Conversly, monoclonal antibodies to SAF-3 that augment DC function, i.e., antigen presentation and stimulation of T cells, may be a useful adjunct therapy for i) patients with cancer that are undergoing cancer vaccine treatment, or ii) other immunocompromised individuals undergoing vaccination for bacterial or viral pathogens. Additionally, these “agonist” mAbs may activate NK cells which would also be beneficial in these patient populations.

DCs used in the assays described herein have been generated ex vivo from purified CD14+ monocytes cultured for 7 days with 40 ng/ml GM-CSF and 20 ng/ml IL-4. These cells are considered “immature” DCs because of their phenotype (CD1a+, CD86^{lo}, Class II^{med}, CCR6+) and function (efficient uptake of antigen, poor T cell stimulation). If 30 ng/ml TNF-α and 10 ng/ml IL-1β are added for the last 48 hours of culture, the DC “mature”. They are now phenotypically CD1a^{lo}, CD86^{hi}, Class II^{hi}, CD83+, CCR6− and CCD7+, and they have lost the ability to capture antigen but they now very efficiently present antigen.

The allogeneic mixed lymphocyte reaction (AlloMLR) was used to asses the productivity of DC:T cell interactions by measuring the proliferation of the T cells. Specifically, 1×10⁵ T cells are mixed with various numbers of DCs, beginning with 50,000 and making serial two fold dilutions ending with 781 per well of a 96 well plate in a total volume of 200 ul of RPMI containing 10% FCS. The cells are cultured for 3, 4 or 5 days, depending on the experiment. Eighteen hours before harvesting the cells each well is pulsed with 1 uCi ³H-thymidine in 50 ul of the above media. The plates are harvested and counted in a TopCount Scintillation counter (Packard). The DCs are generated from Donor A while the T cells are purified from Donor B. Since the donors have different Major Histocompatibility antigens (MHC), the T cells will recognize the foreign MHC and proliferate. The more MHC and co-stimulatory molecules expressed by the DCs, the stronger the proliferative response of the T cells.

Two approaches have been used to evaluate the mAbs for their effect on DCs and the interaction with T cells: 1) addition of the mAb directly to the AlloMLR; and 2) addition of the mAb to the developing DC cultures. A summary of the results from the evaluation of the mAbs directly in the AlloMLR is presented in Table 4.

TABLE 4

| Antibody | n = | Effect |
|---|---|---|
| 16D3 | 9 | small amount of inhibition |
| 2H10 | 5 | no effect |
| 2G4 | 5 | no effect |
| 12E7 | 5 | no effect |
| 16F2 | 7 | consistent inhibition |
| 12B1 | 8 | no effect |
| 13H5 | 5 | small increase in proliferation |
| 13D5 | 7 | consistent inhibition |
| 7D9 | 5 | no effect |

Two of the mAbs, 16F2 and 13D5, that consistently give strong inhibition of the AlloMLR.

Table 5 shows a dose titration of 16F2 and 13D5 with approximately 50% inhibition observed at a dose of 1 ug/ml for 16F2 and 0.3 ug/ml for 13D5.

TABLE 5

| | | T:DC Ratio | | | | | |
|---|---|---|---|---|---|---|---|
| | Conc. | 4:1 | | 16:1 | | 64:1 | |
| Antibody | (ug/ml) | CPM (SD) | % inhib.¹ | CPM (SD) | % inhib.¹ | CPM (SD) | % inhib.¹ |
| 16F2 | 30.0 | 1804 (704) | 83 | 397 (124) | 90 | 192 (50) | 93 |
| | 10.0 | 5512 (1645) | 55 | 2423 (931) | 54 | 850 (246) | 73 |
| | 3.0² | 9109 (1459) | 26 | 2961 (677) | 44 | 1001 (174) | 68 |
| | 1.0² | 7067 (1120) | 42 | 2863 (777) | 46 | 1079 (420) | 65 |
| | 0.3 | 10080 (1954) | 0 | 5086 (1226) | 0 | 2699 (804) | 0 |
| | 0.1 | 11183 (844) | 0 | 5256 (1119) | 0 | 2246 (639) | 0 |
| 13D5 | 30.0 | 1938 (789) | 82 | 659 (239) | 84 | 241 (59) | 92 |
| | 10.0 | 5365 (1047) | 56 | 2284 (543) | 57 | 957 (146) | 69 |
| | 3.0² | 7451 (1198) | 39 | 3568 (955) | 32 | 1812 (259) | 42 |
| | 1.0² | 8433 (965) | 31 | 3649 (209) | 31 | 1835 (140) | 43 |
| | 0.3 | 8795 (688) | 30 | 4208 (644) | 44 | 1722 (326) | 53 |
| | 0.1 | 9497 (1569) | 0 | 5429 (944) | 0 | 2918 (669) | 0 |
| Control³ | 30.0 | 10586 (1474) | | 4072 (1624) | | 2857 (1141) | |
| | 10.0 | 12227 (882) | | 5266 (609) | | 3125 (291) | |
| | 3.0 | 8203 (673) | | 3188 (669) | | 2043 (510) | |
| | 1.0 | 12641 (1984) | | 7532 (1436) | | 3661 (907) | |
| | 0.3 | 10774 (707) | | 5370 (990) | | 2640 (159) | |
| | 0.1 | 11495 (1357) | | 5154 (660) | | 2236 (409) | |

¹Compared to control at the same concentration
²Compared to control at 10 ug/ml
³Isotype matched

One mAb, 16D3, also inhibits but not as well and at a higher concentration of antibody (Table 6).

TABLE 6

| | | T:DC Ratio | | | | | |
|---|---|---|---|---|---|---|---|
| | Conc. | 4:1 | | 16:1 | | 64:1 | |
| Antibody | (ug/ml) | CPM (SD) | % inhib.¹ | CPM (SD) | % inhib.¹ | CPM (SD) | % inhib.¹ |
| 16D3 | 10.0 | 13174 (1238) | 8 | 5383 (130) | 23 | 1678 (333) | 48 |
| Control² | 10.0 | 14334 (2481) | | 7014 (753) | | 3228 (526) | |

¹Compared to control at the same concentration
²Isotype matched

Of particular interest is mAb 13H5 that consistently gave an increase in the T cell proliferation in the AlloMLR (representative data in Table 7).

TABLE 7

| | | T:DC Ratio | | | | | |
|---|---|---|---|---|---|---|---|
| | Conc. | 4:1 | | 16:1 | | 64:1 | |
| Antibody | (ug/ml) | CPM (SD) | % incr.¹ | CPM (SD) | % incr.¹ | CPM (SD) | % incr.¹ |
| 13H5 | 10.0 | 14110 (2873) | 17 | 6934 (2213) | 76 | 2990 (838) | 120 |
| | 3.0 | 14286 (1080) | 17 | 5135 (448) | 28 | 2063 (252) | 57 |
| Control² | 10.0 | 12075 (651) | | 3934 (454) | | 1356 (113) | |
| | 3.0 | 12244 (848) | | 4013 (555) | | 1310 (329) | |

¹Compared to control at the same concentration
²Isotype matched

The T cell proliferation has approximately doubled at two T:DC ratios when the antibody is used at 10 ug/ml.

DCs used for all of the assays described herein have been generated ex vivo from purified CD14+ monocytes cultured for 7 days with 40 ng/ml GM-CSF and 20 ng/ml IL-4. For the last 48 hours, 30 ng/ml TNF-α and 10 ng/ml IL-1β are added to “mature” the DCs. In a first experiment, SAF-3 mAb (clone 12B1) was added to the DC cultures starting at day 0 and re-added when the cells were fed on days 3 and 5. After 7 days of culture, the cells were harvested, analyzed by FACS analysis for their phenotype, and used as stimulator cells in an AlloMLR. As can be seen in Table 8, expression of SAF-3 is now absent on the DCs cultured in the presence of 12B1.

Mean Fluorescent Intensity

| mAb in Culture | Control | Anti-SAF-3¹ |
|---|---|---|
| 12B1 | 3.5 | 3.5 |
| Control | 3.2 | 8.1 |

¹mAb 2H10 was used for FACS analysis

These DC now stimulate T cells very poorly compared to the isotype control (Table 9).

TABLE 9

| | | T:DC Ratio | | | | | |
|---|---|---|---|---|---|---|---|
| | Conc. | 4:1 | | 16:1 | | 64:1 | |
| Antibody | (ug/ml) | CPM (SD) | % inhib.¹ | CPM (SD) | % inhib.¹ | CPM (SD) | % inhib.¹ |
| 12B1 | 10.0 | 7486 (1056) | 23 | 1580 (326) | 67 | 369 (27) | 76 |
| Control² | 10.0 | 9752 (1019) | | 4719 (625) | | 1512 (121) | |

¹Compared to control at the same concentration
²Isotype matched

### Example 3

Cloning and Sequencing of Heavy and Light Chain Antigen Binding Regions

Full-length V_{H} and V_{K} region sequences were obtained for monoclonal antibody 12B1 using the following cloning strategy. The N-terminal amino acid sequences of the mAb 12B1 V_{H} and V_{K} were determined. In the event that the N-terminal V region residue was blocked with pyroglutamic acid, enzymatic de-blocking was performed by means of pyroglutamate aminopeptidase.

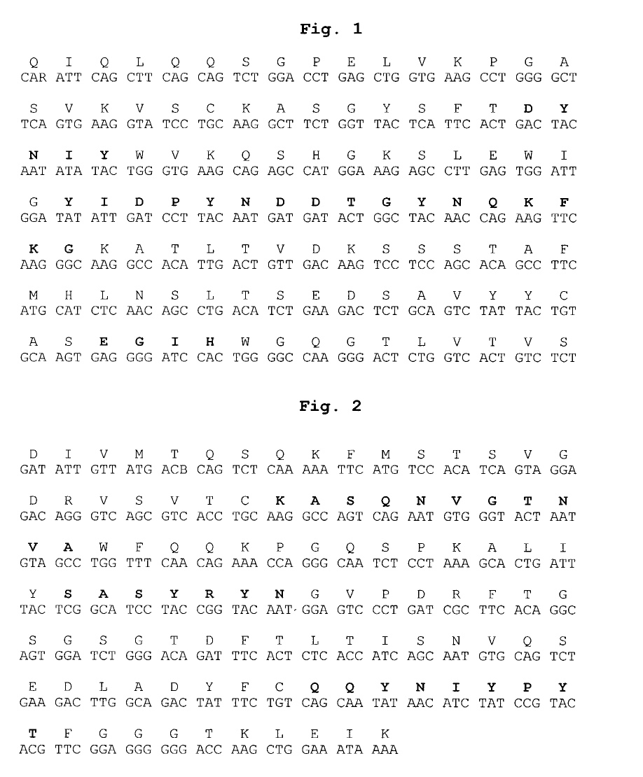

Total hybridoma RNA was purified, reverse transcribed and PCR amplified. For the heavy chains, the RNA/DNA hybrid was PCR amplified using a mouse IgG CH1-specific primer and a degenerate primer based on the N-terminal protein sequence. Similarly, for the light chains, the RNA/DNA hybrid was PCR amplified using a mouse C kappa primer and a degenerate primer based on the N-terminal protein sequence. PCR products of the appropriate size, i.e., ˜350 were cloned into a plasmid vector, and sequenced by a modification of the Sanger method (Sanger et al. (1977) PNAS USA 74:5463). In each case, the sequences of multiple V_{H} clones and the sequences of multiple V_{K} clones were compared to generate a consensus heavy chain variable region sequence and consensus light chain variable region sequence, respectively. The nucleotide and deduced amino acid sequences of the V_{H} and V_{K} regions of monoclonal antibody 12B1 are shown in FIGS. 1 and 2, respectively.

What is claimed is:

1. A monoclonal antibody that binds to human SAF-3.

2. The antibody of claim 1 wherein the antibody has the identifying characteristics of monoclonal antibody that is a member selected from the group consisting of 12B1, 2H10, 2G4, 7D9, 13H5, 16F2, 13D5, 16D3 and 12E7.

3. The antibody of claim 2, wherein the antibody is monoclonal antibody 12B1.

4. The antibody of claim 2, wherein the antibody is monoclonal antibody 13H5.

5. An isolated polypeptide comprising an immunoglobulin complementarity determining region of the antibody of claim 1.

6. An isolated polypeptide comprising an immunoglobulin complementarity determining region of the antibody of claim 2.

7. An isolated polypeptide comprising an immunoglobulin complementarity determining region of the antibody of claim 3.

8. An isolated polypeptide comprising an immunoglobulin complementarity determining region of the antibody of claim 4.

9. An isolated polynucleotide encoding the polypeptide of claim 5.

10. An isolated polynucleotide encoding the polypeptide of claim 6.

11. An isolated polynucleotide encoding the polypeptide of claim 7.

12. An isolated polynucleotide encoding the polypeptide of claim 8.

13. The polypeptide of claim 7 wherein the immunoglobulin complementarity determining region that comprises the polypeptide is set forth in a member of the group consisting of SEQ ID NO:9, 10, 11, 12, 13 and 14.

14. The polypeptide of claim 13 wherein the immunoglobulin complementarity determining region comprises the polypeptides set forth in SEQ ID NOs:9, 10 and 11.

15. The polypeptide of claim 13 wherein the immunoglobulin complementarity determining region comprises the polypeptides set forth in SEQ ID NOs:12, 13 and 14.

16. An isolated polynucleotide encoding polypeptide of claim 13.

17. An isolated polynucleotide encoding polypeptide of claim 14.

18. An isolated polynucleotide encoding polypeptide of claim 15.

19. The antibody of claim 1 wherein the immunoglobulin complementarity determining region of the antibody comprises the polypeptides set forth in SEQ ID NO:9, 10, 11, 12, 13 and 14.

20. The antibody of claim 19 comprising a heavy chain variable region polypeptide as set forth in SEQ ID NO:6 and a kappa light chain variable region polypeptide as set forth in SEQ ID NO:8.

21. An isolated polynucleotide encoding a polypeptide comprising a member selected from the group consisting of SEQ ID NO:6 and SEQ ID NO:8.

22. A hybridoma cell line that produces a monoclonal antibody having the identifying characteristics the monoclonal antibody 12B1.

23. A hybridoma cell line that produces a monoclonal antibody having the identifying characteristics the monoclonal antibody 13H5.

24. A pharmaceutical composition comprising the monoclonal antibody of claim 1.

25. A pharmaceutical composition comprising the monoclonal antibody of claim 2.

26. A pharmaceutical composition comprising the monoclonal antibody of claim 3.

27. A pharmaceutical composition comprising the monoclonal antibody of claim 4.

28. A method for detecting the presence of a cell in a sample wherein the cell comprises an SAF-3 protein, the method comprising:
a) exposing the sample to an antibody that binds to SAF-3; and
b) detecting the antibody that is bound to SAF-3.

29. The method of claim 28 wherein the sample is treated before exposure to the antibody such that the SAF-3 protein accessible to binding by the antibody.

30. The method of claim 28 wherein the antibody has the identifying characteristics of monoclonal antibody 12B1.

31. The method of claim 30 wherein the antibody is monoclonal antibody 12B1.

32. The method of claim 28 wherein the antibody has the identifying characteristics of monoclonal antibody 13H5.

33. The method of claim 32 wherein the antibody is monoclonal antibody 13H5.

34. A method for treating or preventing cancer, inflammation, autoimmunity, allergy, asthma, rheumatoid arthritis, CNS inflammation, multiple sclerosis, AIDS, and bacterial, fungal, protozoan and viral infections in a mammal comprising administering an effective dose of the monoclonal antibody of claim 1.

35. The method of claim 34 wherein the monoclonal antibody that is administered has the identifying characteristics of a monoclonal antibody that is a member selected from the group consisting of 12B1, 2H10, 2G4, 7D9, 13H5, 16F2, 13D5, 16D3 and 12E7.

36. A method for modulating an immune response in a mammal comprising administering an effective dose of the monoclonal antibody of claim 1.

37. The method of claim 36 wherein the immune response is downregulated.

38. The method of claim 37 wherein the monoclonal antibody that is administered has the identifying characteristics of a monoclonal antibody that is a member selected from the group consisting of 16D3, 16F2 and 13D5.

39. The method of claim 36 wherein the immune response is enhanced.

40. The method of claim 39 wherein the monoclonal antibody that is administered has the identifying characteristics of monoclonal antibody 13H5.


SEQUENCE LISTING (14 sequences)

SEQ ID NO: 1 - DNA, 1501 bp, Human
cctggcacctccaaccccagatatgctgctgctgctgctgctgcccctgctctgggggagggagagggtggaaggacagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacagccttgacccacaggcccaacatccttatccccggtaccctggagtctggctgcttccagaatctgacctgctctgtgccctgggcctgtgagcaggggacgccccctatgatctcctggatggggacctctgtgtcccccccgcacccctccaccacccgctcctcggtgctcaccctcatcccacagccccagcaccacggcaccagcctcacctgtcaggtgaccttgcctggggccggcgtgaccacgaacaggaccatccaactcaatgtgtcctaccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggagtgttgctgggggcggtcgggggagctggagccacagccctggtcttcctctccttctgtgtcatcttcattgtagtgaggtcctgcaggaagaaatcggcaaggccagcagcggacgtgggagacgtaggcatgaaggatgcaaacaccatcaggggctcagcctctcagggtaacctgactgagtcctgggcagatgataacccccgacaccatggcctggctgcccactcctcaggggaggaaagagagatccagtatgcacccctcagctttcataagggggagcctcaggacctatcaggtcaagaagccaccaacaatgagtactcagagatcaagatccccaagtaagaaaatgcagaggctcgggcttgtttgagggttcacgacccctccagcaaaggagtctgaggctgattccagtag

SEQ ID NO: 2 - protein, 467 aa, Human
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPPHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDVGMKDANTIRGSASQGNLTESWADDNPRHHGLAAHSSGEEREIQYAPLSFHKGEPQDLSGQEATNNEYSEIKIPK

SEQ ID NO: 3 - DNA, 1502 bp, Human
ggcacgaggcagttcctgagagaagaaccctgaggaacagacgttccctcgcggccctggcacctccaaccccagatatgctgctgctgctgctgctgcccctgctctgggggagggagagggtggaatggcagaagagtaaccggaaggattactcgctgacgatgcagagttccgtgaccgtgcaagagggcatgtgtgtccatgtgcgctgctccttctcctacccagtggacagccagactgactctgacccagttcatggctactggttccgggcagggaatgatataagctggaaggctccagtggccacaaacaacccagcttgggcagtgcaggaggaaactcgggaccgattccacctccttggggacccacagaccaaaaattgcaccctgagcatcagagatgccagaatgagtgatgcggggagatacttctttcgtatggagaaaggaaatataaaatggaattataaatatgaccagctctctgtgaacgtgacataccctcctcagaacttgactgtgactgtcttccaaggagaaggcacagcatccacagctctggggaacagctcatctctttcagtcctagagggccagtctctgcgcttggtctgtgctgttgacagcaatccccctgccaggctgagctggacctggaggagtctgaccctgtacccctcacagccctcaaaccctctggtactggagctgcaagtgcacctgggggatgaaggggaattcacctgtcgagctcagaactctctgggttcccagcacgtttccctgaacctctccctgcaacaggagtacacaggcaaaatgaggcctgtatcaggagtgttgctgggggcggtcgggggagctggagccacagccctggtcttcctctccttctgtgtcatcttcattgtagtgaggtcctgcaggaagaaatcggcaaggccagcagcggacgtgggagacataggcatgaaggatgcaaacaccatcaggggctcagcctctcagggtaacctgactgagtcctgggcagatgataacccccgacaccatggcctggctgcccactcctcaggggaggaaagagagatccagtatgcacccctcagctttcataagggggagcctcaggacctatcaggtcaagaagccaccaacaatgagtactcagagatcaagatccccaagtaagaaaatgcagaggctcgggcttgtttgagggttcacgacccctccagcaaaggagtctgaggctgattccagtagaattagcagccctcaatgctgtgcaacaagacatcagaacttattcctcttgtctaactgaaaatgcatgcctgatgaccaaactctccctttccccatccaatcggtccacactccccgccctggcctttggtacccaccattctcctctgtacttctctaaggatgactactttagattccgaatatagtgagattgtaacgtgaaaaaaaaaaaaaaaaaaa

SEQ ID NO: 4 - protein, 374 aa, Human
MLLLLLLPLLWGRERVEWQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 5 - DNA, 336 bp, Human
carattcagcttcagcagtctggacctgagctggtgaagcctggggcttcagtgaaggtatcctgcaaggcttctggttactcattcactgactacaatatatactgggtgaagcagagccatggaaagagccttgagtggattggatatattgatccttacaatgatgatactggctacaaccagaagttcaagggcaaggccacattgactgttgacaagtcctccagcacagccttcatgcatctcaacagcctgacatctgaagactctgcagtctattactgtgcaagtgaggggatccactggggccaagggactctggtcactgtctct

SEQ ID NO: 6 - protein, 112 aa, Human
QIQLQQSGPELVKPGASVKVSCKASGYSFTDYNIYWVKQSHGKSLEWIGYIDPYNDDTGYNQKFKGKATLTVDKSSSTAFMHLNSLTSEDSAVYYCASEGIHWGQGTLVTVS

SEQ ID NO: 7 - DNA, 321 bp, Human
gatattgttatgacbcagtctcaaaaattcatgtccacatcagtaggagacagggtcagcgtcacctgcaaggccagtcagaatgtgggtactaatgtagcctggtttcaacagaaaccagggcaatctcctaaagcactgatttactcggcatcctaccggtacaatggagtccctgatcgcttcacaggcagtggatctgggacagatttcactctcaccatcagcaatgtgcagtctgaagacttggcagactatttctgtcagcaatataacatctatccgtacacgttcggaggggggaccaagctggaaataaaa

SEQ ID NO: 8 - protein, 107 aa, Human
DIVMTQSQKFMSTSVGDRVSVTCKASQNVGTNVAWFQQKPGQSPKALIYSASYRYNGVPDRFTGSGSGTDFTLTISNVQSEDLADYFCQQYNIYPYTFGGGTKLEIK

SEQ ID NO: 9 - protein, 5 aa, Human
DYNIY

SEQ ID NO: 10 - protein, 17 aa, Human
YIDPYNDDTGYNQKFKG

SEQ ID NO: 11 - protein, 4 aa, Human
EGIH

SEQ ID NO: 12 - protein, 11 aa, Human
KASQNVGTNVA

SEQ ID NO: 13 - protein, 7 aa, Human
SASYRYN

SEQ ID NO: 14 - protein, 9 aa, Human
QQYNIYPYT